SEQUENCE LISTING (2 sequences)

SEQ ID NO: 1 - protein, 320 aa, Bos taurus
AQVLRGTVADFPGFDERADAETLRKAMKGLGTDEESILTLLTSRSNAQRQEIAVAFKTLFGRDLLDDLKSELTGKFEKLIVALMKPSRLYDAYELKHALKGAGTDEKVLTEIIASRTPEELRAIKQVYEEEYGSSLEDDVVGDTSGYYQRMLVVLLQANRDPDARIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFGTSLYSMIKGDTSGDYKKALLLLCGGEDD

SEQ ID NO: 2 - protein, 320 aa, Bos taurus
AQVLRGTVADFPGFDERADAETLRKAMKGLGTDEETILTLLTSRSNAQRQEIAVAFKTLFGRDLLDDLKSELTGKFEKLIVALMKPSRLYDAYELKHALKGAGTDEKVLTEIIASRTPEELRAIEQVYEEEYGSSLEDDVVGDTSGYYQRMLVVLLQANRDPDARIDEAQVEQDAQALFQAGELKWGTDEEKFITIFGTRSVSHLRRVFDKYMTISGFQIEETIDRETSGNLEQLLLAVVKSIRSIPAYLAETLYYAMKGAGTDDHTLIRVVVSRSEIDLYNIRKEFRKNFGTSLYSMIKGDTSGDYKKALLLLCGGEDD

(19) United States
(12) Patent Application Publication
(10) Pub. No.: US 2011/0236905 A1
(43) Pub. Date: Sep. 29, 2011

(54) METHOD FOR SCREENING CATTLE, CATTLE SCREENED, AND CATTLE SCREENING KIT

(76) Inventors: Kazuya Matsumoto, Wakayama (JP); Kouichi Morimoto, Osaka (JP); Haruka Ikegami, Wakayama (JP); Kouhei Nagai, Kanagawa (JP)

(21) Appl. No.: 12/936,569

(22) PCT Filed: Mar. 4, 2009
(86) PCT No.: PCT/JP2009/054045

§ 371 Date: Oct. 6, 2010

(30) Foreign Application Priority Data
Apr. 8, 2008 (JP) 2008-100138

(51) Int. Cl. G01N 33/566; C12Q 1/02

(52) U.S. Cl. 435/7.1; 435/29

(57) ABSTRACT

There are provided a method for screening individual cattle having useful economic traits in which proteins related to the economic traits of beef cattle are identified by proteomics and are used as biomarkers, and a cattle screening kit for use in the method.

The method for screening individual cattle according to the present invention includes the steps of (1) harvesting body tissue from the cattle, (2) extracting the full complement of proteins from the body tissue harvested, (3) detecting a wild-type annexin A5 protein, an isoform of the wild-type annexin A5 protein, or a modified protein of the wild-type annexin A5 protein or the isoform thereof in the full complement of proteins extracted, and (4) determining whether or not the cattle will have a high average carcass weight on the basis of the type of annexin A5 detected. The wild-type annexin A5 protein, the isoform thereof, and the modified protein thereof are detected by two-dimensional electrophoresis or an antigen-antibody reaction.

### TECHNICAL FIELD

The present invention relates to a method for screening cattle having excellent economic traits, such as carcass weight, cattle screened, and a screening kit for use in the method.

### BACKGROUND ART

Now, the breeding improvement of beef cattle, particularly Japanese Black cattle, which is a meat breed indigenous to Japan, is performed by using a statistical genetic breed improvement method for estimating the genetic capacity of cattle in accordance with a statistical genetic analysis based on accumulated phenotypic information and pedigree information. Establishment of the breed improvement method contributes greatly to improvements in the genetic capacity of Japanese Black cattle.

However, the statistical genetic breed improvement method estimates the probability that progeny will inherit the superior genotype of beef cattle, and cannot provide genetic information about the economic traits of individual beef cattle, such as marbling, carcass weight, rib-eye area, and rib thickness.

Also, the economic traits are often affected by the environment as well as genetic factors. It is therefore difficult to strictly select superior individuals on the basis of the genetic factors. Furthermore, since the breed improvement method based on the statistical genetic method involves mating, fattening, and other processes, breeding in the breed improvement method incurs considerable costs and time.

Accordingly, a breeding method utilizing genes, more specifically, a quantitative trait locus (QTL) analysis has been performed since the latter half of the 1980s. The QTL analysis determines the number of genes related to quantitative traits, such as economic traits, of beef cattle and their positions on a linkage map. This analysis method is expected to bring revolutionary outcomes in the identification of genes responsible for the economic traits of beef cattle. Studies are advancing to develop a breeding method utilizing a bovine DNA marker (see Non-patent Documents 1 and 2).

Although this method has contributed to the identification of causative genes of recessive hereditary diseases, such as claudin-16 deficiency, genes responsible for economic traits have not yet been identified by this method, and a breed improvement method of beef cattle utilizing the responsible genes has not yet been established.

Also, it has recently been believed that the phenotype of an economic trait is related to not only a responsible gene but also a gene for regulating gene expression and epigenetic modification, such as methylation, of DNA and that the analysis must take these factors into account. Furthermore, the QTL analysis has difficulty in selecting a suitable cross parent and involves a family breeding process. As in the statistical genetic method, breeding in the QTL analysis therefore incurs considerable costs and time.

Meanwhile, the present general trends in the studies of life sciences are focused on studies subsequent to genomics (post-genomics), more specifically, studies utilizing analytical methods such as transcriptomics, proteomics, and metabolomics, particularly proteomics. Proteomics comprehensively and systematically analyzes the functions of proteins and their relationships. Research and development are advancing in proteomics.

The reason for this is that protein molecules, which are end products of genes, directly perform physiological functions, and their expression levels and proteins after molecular modification are directly related to the physiological functions at the cellular, tissue, and individual levels. Another reason is that proteomics obviates the necessity for time-consuming expensive processes, such as mating, breeding, and family breeding.

Proteomics has been widely applied to various fields and has advanced particularly in the medical field. For example, in a disease other than genetic diseases, which has a distant causal relationship between a gene mutation and the pathological condition, a change in a protein function in a cellular process is thought to directly trigger the onset of the disease. Thus, identification and development of a biomarker that can trap a disease-associated protein to detect the disease and diagnose the pathological condition have been actively conducted by proteomics (see Patent Documents 1 to 4 and Non-patent Document 3).

However, the animal husbandry field lags far behind in the application of proteomics. Proteins related to the economic traits of beef cattle have not been identified, and these proteins are not used to screen cattle having useful economic traits.

### [Prior Art Documents]

[Patent Document 1] Japanese Unexamined Patent Application Publication No. 2006-308533

[Patent Document 2] Japanese Unexamined Patent Application Publication No. 2007-93597

[Patent Document 3] Japanese Unexamined Patent Application Publication No. 2007-139742

[Patent Document 4] Japanese Unexamined Patent Application Publication No. 2007-523346

[Non-patent Document 1] Annual Report, Shirakawa Institute of Animal Genetics (No. 11), Japan Livestock Technology Association, Shirakawa Institute of Animal Genetics, 2004

[Non-patent Document 2] Nikuyogyu Idensigen Katuyotaisei Seibijigyo Hokokusyo, Japan Livestock Technology Association, 2005

[Non-patent Document 3] “Sikkan Puroteomikusu no Saizensen”, Medical Do, 2005

### SUMMARY OF INVENTION

It is an object of the present invention to provide a method for screening individual cattle having useful economic traits in which proteins related to the economic traits of beef cattle are identified by proteomics and are used as biomarkers, cattle screened by this method, and a cattle screening kit for use in the method.

The present inventors have comprehensively investigated the correlation between proteins in white adipose tissue and economic traits of cattle using two-dimensional electrophoresis, mass spectrometry, and data analysis by a statistical method in combination to search for proteins usable as biomarkers related to the economic traits of the cattle. As a result, the present inventors made the present invention by finding a significant correlation between the expression of particular proteins and the carcass weight.

Specifically, a method for screening cattle according to Claim 1 of the present invention includes the steps of (1) harvesting body tissue from the cattle, (2) extracting the full complement of proteins from the body tissue harvested, (3) detecting a wild-type annexin A5 protein, an isoform of the wild-type annexin A5 protein, or a modified protein of the wild-type annexin A5 protein or the isoform thereof in the full complement of proteins extracted, and (4) determining whether or not the cattle from which the body tissue has been harvested has a high average carcass weight on the basis of whether any one of the wild-type annexin A5 protein and modified protein thereof, an isoform of the wild-type annexin A5 protein and modified protein thereof, or both of them, are detected in the detecting step.

It should be noted that Annexin A5 (also referred to as annexin V or ANX5) belongs to an annexin family of calcium/phospholipid-binding proteins. Annexin A5 is known to be involved in various physiological phenomena, such as cell-spreading promoting action, gonadotropin production promoting action, and apoptosis-suppressing action.

Also, a wild-type annexin A5 protein is a protein (CaBP33) having an amino acid sequence of SEQ ID No. 1. An isoform of the wild-type annexin A5 protein has a different function and a different amino acid sequence from the wild-type protein and, more specifically, is a protein (CaBP37) having an amino acid sequence of SEQ ID No. 2. A modified protein is a protein subjected to a chemical modification, such as glycosylation, phosphorylation, acetylation, or methylation.

A method for screening cattle according to Claim 2 of the present invention is the method for screening cattle according to Claim 1, wherein the wild-type annexin A5 protein, the isoform of the wild-type annexin A5 protein, and the modified protein of the wild-type annexin A5 protein or the isoform thereof are detected by electrophoresis in the detecting step.

A method for screening cattle according to Claim 3 of the present invention is the method for screening cattle according to Claim 1, wherein the wild-type annexin A5 protein, the isoform of the wild-type annexin A5 protein, and the modified protein of the wild-type annexin A5 protein or the isoform thereof are detected by an antigen-antibody reaction in the detecting step.

Cattle and dressed carcass according to Claim 4 of the present invention are cattle screened by the method for screening cattle according to Claims 1 to 3. Cattle according to Claim 5 is progeny or clone of the cattle according to Claim 4. Dressed carcass according to Claim 6 is dressed carcass of the cattle according to Claim 4 or 5.

A cattle screening kit according to Claim 7 of the present invention includes an antibody that specifically bonds to a wild-type annexin A5 protein or a modified protein thereof and an antibody that specifically bonds to an isoform of the wild-type annexin A5 protein or a modified protein thereof.

By using a method for screening cattle and a screening kit according to the present invention, it was able to easily determine cattle having a high carcass weight. The difference in carcass weight between cattle having a high carcass weight and cattle having a low carcass weight as determined by the method for screening cattle ranges from approximately 20 to 36 kg. Assuming that the price of dressed carcass is 2000 yen/kg on average, it was found that the method for screening cattle produces an economic effect ranging from 40 to 70 thousand yen per head of cattle and is expected to increase the income of a general fattening farm (50 to 100 heads of cattle) by 2 to 7 million yen. This can improve productivity in animal husbandry and improve and stabilize the lives of farms.

### BRIEF DESCRIPTION OF DRAWINGS

FIG. 1 shows results of a two-dimensional electrophoresis of a full complement of proteins in white adipose tissue of cattle.

FIG. 2 is a fragmentary enlarged view of FIG. 1.

FIG. 3 is a scatter diagram of a relationship between a type of annexin A5 in white adipose tissue of cattle and carcass weight.

### DESCRIPTION OF EMBODIMENTS

### 1. Screening Method

A method for screening cattle according to the present invention includes the steps of (1) harvesting body tissue from the cattle, (2) extracting the full complement of proteins from the body tissue harvested, (3) detecting an annexin A5-associated protein in the full complement of proteins extracted, and (4) determining whether or not the cattle will have a high average carcass weight on the basis of the type of annexin A5 detected. Each of the steps will be described in detail below.

### (1) Harvesting Step

The harvesting step is a step of harvesting body tissue. Body tissue to be harvested may be any body tissue expressing annexin A5, and more specifically, includes white adipose tissue, muscular tissue, skin tissue, and blood. Blood serum is particularly preferred because harvesting blood serum minimally invades a specimen.

Body tissue can be harvested by any known method without limitation. Specific examples include suction with a syringe, surgery under local anesthesia, and liposuction. Liposuction is generally employed in cosmetic surgery and more specifically includes ultrasonic liposuction, powered liposuction with a cannula, and syringe liposuction.

### (2) Extracting Step

The extracting step is a step of extracting the full complement of proteins from body tissue. Any known method can be used without limitation to extract the full complement of proteins in quantity and quality usable in the detecting step described below. In a specific example, body tissue is put in a buffer solution having an appropriate pH. Tissue and cells are crushed in a homogenizer and are centrifuged to yield supernatant. If necessary, treatment with an enzyme or an organic solvent may be performed.

### (3) Detecting Step

The detecting step is a step of detecting a wild-type annexin A5 protein, an isoform of the wild-type annexin A5 protein, a modified protein of the wild-type annexin A5 protein, and a modified protein of the isoform of the wild-type annexin A5 protein in the full complement of proteins extracted, for example, by 1) electrophoresis or 2) an antigen-antibody reaction. The detecting step will be further described below.

### 1) Detection by Electrophoresis

Detection by electrophoresis may be performed by any known method for separating proteins by the difference in the electric charge or isoelectric point of the proteins and detecting the wild-type annexin A5 protein and the like. Specific examples include (a) detection by one-dimensional electrophoresis and (b) detection by two-dimensional electrophoresis described below.

### (a) Detection by One-Dimensional Electrophoresis

In detection by one-dimensional electrophoresis, for example, proteins are separated by electrophoresis with a native gel or an SDS polyacrylamide gel, are transferred to a nitrocellulose film, and then, are detected by Western blotting, which uses antibodies to detect proteins.

Examples of the antibodies include monoclonal antibodies, polyclonal antibodies, single-chain antibodies, humanized antibodies, chimeric antibodies, and bifunctional antibodies, which can simultaneously recognize two epitopes. These antibodies can be produced by administering a wild-type annexin A5 protein, an isoform thereof, a modified protein thereof, or a fragment thereof to an animal other than human by a common protocol, such as a hybridoma technique.

The antibodies may be labeled with a fluorescent substance, such as fluorescein isocyanate (FITC) or tetramethylrhodamine isocyanate, a radioisotope, such as ¹²⁵I, ³²P, ¹⁴C, ³⁵S, or ³H, or an enzyme, such as alkaline phosphatase, peroxidase, β-galactosidase, or phycoerythrin, or may be fused with a fluorescent protein, such as a green fluorescent protein (GFP).

### (b) Detection by Two-Dimensional Electrophoresis

Detection by two-dimensional electrophoresis is performed by two-dimensional electrophoresis and the analysis of the electrophoretic pattern. A two-dimensional electrophoresis method for use in the present invention may be any known method, without limitation, provided that two physical properties, isoelectric point and molecular weight, of a protein are utilized for separation. Specifically, after one-dimensional isoelectric focusing with a capillary gel or a strip gel, the gel is placed on an SDS-polyacrylamide gel (SDS-PAGE) or an agarose gel and is electrophoresed perpendicularly to the migration direction of the isoelectric focusing.

The analysis of a two-dimensional electrophoretic pattern involves staining a gel by a known method, such as coomassie brilliant blue (CBB), SYPRO Ruby (registered trademark), or silver staining, capturing an image of the stained gel on a computer with a scanner or a CCD camera, reducing noise or correcting the background of the captured image, and detecting protein spots of a wild-type annexin A5 protein, an isoform of the wild-type annexin A5 protein, and modified proteins of these proteins appearing at a pH in the range of 4.0 to 5.0 and a molecular weight in the range of approximately 30 to 40 KDa.

### 2) Detection by Antigen-Antibody Reaction

Detection by an antigen-antibody reaction may be a known immunological detection method that uses an antibody that can specifically bond to a wild-type annexin A5 protein and an antibody that can specifically bond to an isoform of the wild-type annexin A5 protein and modified proteins of these proteins. Specific examples include ELISA, RIA, a fluorescent antibody technique, and immunohistochemical analysis. Note that antibodies used in the detection by an antigen-antibody reaction are the same antibodies as used in the one-dimensional electrophoresis.

### (4) Determining Step

The determining step is a step of determining the economic traits of cattle of interest on the basis of the type of annexin A5 detected in the detecting step. Specifically, detection of any one of a wild-type annexin A5 protein (CaBP33) and modified protein thereof, an isoform of the wild-type annexin A5 protein (CaBP37) and modified protein thereof (the case of homo)-indicates that the cattle will have a high average carcass weight. On the contrary, detection of both a wild-type annexin A5 protein and modified protein thereof, and the isoform and modified protein thereof (the case of hetero) indicates that the cattle will have a low average carcass weight.

### 2. Cattle

Cattle according to the present invention includes cattle screened by a screening method according to the present invention, progeny thereof, and clone cattle thereof. Progeny and clone cattle are produced by sexual reproduction and asexual reproduction, for example, a known method, such as a “fertilized egg splitting method” or a “nuclear transfer technique”. In the “fertilized egg splitting method”, one fertilized egg is divided into two to four pieces, and the pieces are transplanted into surrogate cows to produce cattle. In the “nuclear transfer technique”, a nuclear of a fertilized egg or a somatic cell is transplanted into an unfertilized egg to produce a “clone egg”, which in turn is transplanted into a surrogate cow to produce cattle.

### 3. Dressed Carcass of Cattle

Dressed carcass of cattle according to the present invention includes dressed carcass of cattle screened by a screening method according to the present invention, progeny thereof, and clone cattle thereof.

### 4. Screening Kit

Antibodies and buffer solutions required for the detection of a wild-type annexin A5 protein, an isoform of the wild-type annexin A5 protein, and modified proteins of these proteins by an antigen-antibody reaction may be commercially available products. These products may be separately bought and used. However, a kit containing these products can obviate the necessity of buying each component separately, facilitating the detection by an antigen-antibody reaction. A kit further containing a buffer solution necessary to extract a protein can further facilitate the determination of the economic traits of cattle.

While the present invention will be further described in the following examples, these examples do not limit the claims of the present invention by any means.

### Example 1

### 1. Search for Proteins Usable as Biomarkers

The full complement of proteins (proteome) were extracted from a plurality of bovine white adipose tissue samples of a known lineage and quality. The analysis of correlation between the proteome and the trait, such as the quality of meat, of each individual was performed to search for proteins usable as biomarkers. The details will be described below. Unless otherwise specified, % refers to % by volume.

### (1) Protein Extraction

Bovine white adipose tissue samples used were approximately 150 white adipose tissue samples that were harvested from Hida-gyu (Japanese black cattle) having known economic traits, such as lineage, carcass weight, rib-eye area, and rib thickness, and that were cryogenically-preserved in Gifu Prefectural Livestock Research Institute.

The full complement of proteins were extracted as described below. First, a protease inhibitor (Complete Mini, manufactured by F. Hoffmann-La Roche Ltd.) was added to a solution containing 420 mg/ml urea, 140.3 mg/ml thiourea, 40 mg/ml CHAPS, a 0.5% IPG buffer solution (IPG Buffer pH 3-11 NL, manufactured by GE Healthcare), 0.05% Tributylphosphin (hereinafter abbreviated as TBP), and 0.1 mg/ml bromophenol Blue (hereinafter abbreviated as BPB) in accordance with the instructions of the provider to prepare an extraction solution. 1 ml of the extraction solution was added to 1 g of adipose tissue and was homogenized.

Then, the homogenized suspension was centrifuged to remove a precipitate. The supernatant was collected as a sample. Finally, Protein Assay (Bio-Rad Laboratories, Inc.) and an absorptiometer (UV mini 1240, manufactured by Shimadzu Co.) were used to measure the concentration of the full complement of proteins by a modified Bradford-HCl assay (see Electrophoresis 1985, 6, 559-563). The measurement results showed that 3.5 mg of proteins on average were extracted from 1 g of the adipose tissue. Bovine γ-globulin was used as a standard protein, and the absorbance at 595 nm was measured.

### (2) Two-Dimensional Electrophoresis

### 1) First Electrophoresis (Isoelectric Focusing)

The sample prepared in (1) was mixed and diluted in a swelling liquid (containing 420 mg/ml urea, 140.3 mg/ml thiourea, 40 mg/ml CHAPS, a 0.5% IPG buffer solution (IPG Buffer pH 3-11 NL (manufactured by GE Healthcare), 0.05% TBP, and 0.1 mg/ml BPB) to a protein concentration of 0.375 mg/ml. 400 μl of the swelling liquid containing the sample (150 μg of proteins) was injected into a swelling tray (manufactured by GE Healthcare) and was covered with a DryStrip gel (Immobiline DryStrip pH 3-11 NL 18 cm, manufactured by GE Healthcare).

In order to prevent the DryStrip to be dried, the DryStrip was covered with mineral oil (Immobiline DryStrip Cover Fluid, manufactured by GE Healthcare). The swelling tray was covered and was left still for six hours or more to swell the DryStrip. The DryStrip was placed in Multiphor II (manufactured by GE Healthcare) and was subjected to isoelectric focusing at 15° C. and 26.8 kVh.

### 2) Second Electrophoresis (SDS-PAGE)

After the completion of the isoelectric focusing, the DryStrip was immersed in an SDS equilibration buffer (a) (containing 6.057 mg/ml Tris-HCl (pH 8.8), 360.4 mg/ml Urea, 30% glycerol, 20 mg/ml SDS, and 10 mg/ml DTT) for 15 minutes. After the SDS equilibration buffer (a) was discarded, the DryStrip was immersed in an SDS equilibration buffer (b) (containing 6.057 mg/ml Tris-HCl (pH 8.8), 360.4 mg/ml Urea, 30% glycerol, 20 mg/ml SDS, and 25 mg/ml iodo acetamide) for 15 minutes for equilibration. The equilibrated DryStrip was placed on an SDS-PAGE gel (gel concentration: 10%).

The gel was placed in a slab electrophoresis apparatus (Tetra-200, manufactured by Anatech Co., Ltd.) and was filled with a running buffer (3 mg/mg tris, 14.4 mg/ml glycine, and 1 mg/ml SDS). Electrophoresis was performed at 30 mA for four hours per sheet of gel until a BPB band was observed at the lower end of the gel.

### 3) Staining

After the completion of the electrophoresis, the gel was transferred into a plastic container. A fixative (10% methanol and 7% aqueous acetic acid) was added to the plastic container such that the gel was sufficiently immersed in the fixative. The gel was gently shaken at room temperature for approximately 30 minutes. The fixative was replaced with a new fixative, and the gel was gently shaken at room temperature for another approximately 30 minutes.

The fixative was removed from the plastic container. A SYPRO Ruby stain solution (registered trademark, manufactured by Molecular Probes, Inc.) was added to the plastic container. The plastic container was entirely covered with aluminum foil for shading. The plastic container was gently shaken at room temperature for approximately 12 hours.

The stain solution was removed from the plastic container. A decolorant (10% ethanol) was added to the plastic container such that the gel was sufficiently immersed in the decolorant. The plastic container was entirely covered with aluminum foil for shading. The plastic container was gently shaken at room temperature for approximately 30 minutes. The decolorant was replaced with a new decolorant. The stained gel was stored in the decolorant until the stained gel was used for image analysis described below.

### (3) Image Analysis

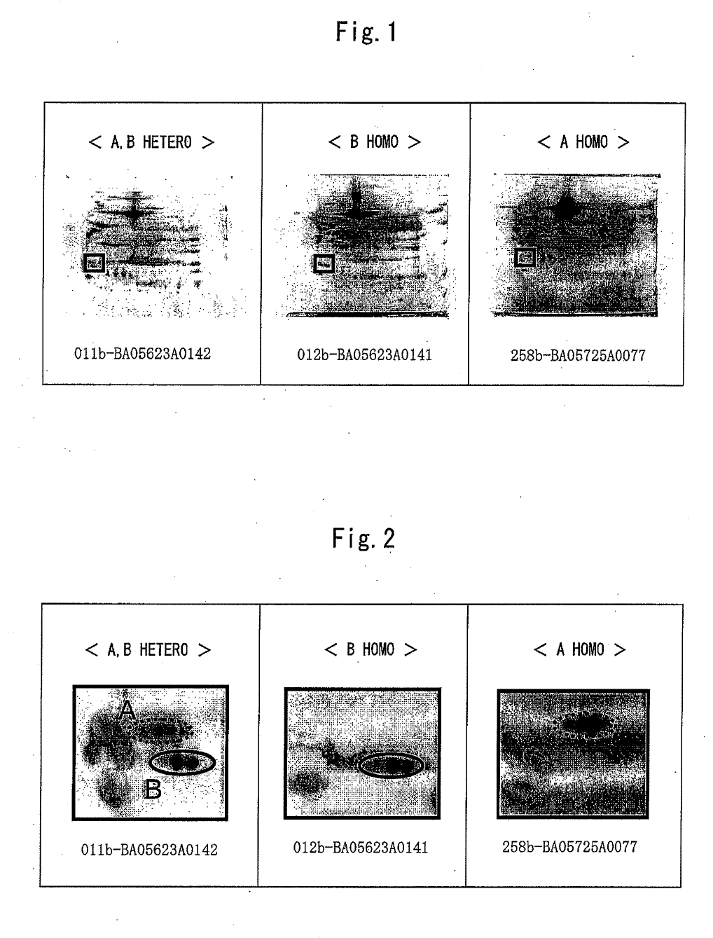

Electrophoresis images of the stained gel were captured with a gel imaging system (AlphaImager, manufactured by Alpha Innotech Co.). FIG. 1 shows parts of the captured electrophoresis images. FIG. 2 is a fragmentary enlarged view of a square in FIG. 1. Alphanumeric characters under the electrophoresis images are identification codes for each individual.

The captured images were corrected for a distortion with an image analysis software (Progenesis TT900, manufactured by PerkinElmer, Inc.). Another image analysis software (Progenesis PG220, manufactured by PerkinElmer, Inc.) was used for the detection of protein spots, matching of protein spots between gels (individuals), quantitative determination of spots, and comparison of quantitative values between gels.

### Example 2

### 2. Identification of Proteins

A plurality of protein spots (approximately 350 spots) including encircled protein spots in FIG. 2 were extracted from stained gels after two-dimensional electrophoresis and were identified by mass spectrometry. The following were specific procedures.

(1) Extraction from Gel

A portion containing a particular protein spot was cut from a gel with tweezers, was placed in a well of a 96-well MTP plate, and was immersed in 0.1 ml of a decolorant A (a mixture of methanol and 100 mM aqueous ammonium hydrogen carbonate in equal proportions) three times for 20 minutes each. After the decolorant A was removed, the portion of the gel was immersed in 0.1 ml of 100% acetonitrile for 5 minutes. Acetonitrile was evaporated to completely dry the gel. 30 μl of a trypsin solution (0.83 μg/ml trypsin (Sequencing grade Trypsin, manufactured by Promega Co.) and 25 mM ammonium hydrogen carbonate) was added to the dried gel. The gel was allowed to react overnight at 30° C. to produce an extract.

### (2) Desalination

A ZipTipμC18 pipette chip (registered trademark, manufactured by Nihon Millipore K.K.) was attached to the tip of a micropipette. 90% aqueous acetonitrile was pipetted several times to wash the pipette chip. 0.1% aqueous trifluoroacetic acid (hereinafter abbreviated as TFA) was pipetted several times to equilibrate the pipette chip. The extract was pipetted several times with the washed and equilibrated pipette chip to bond proteins in the extract to a resin in the pipette chip. A cleaning liquid (0.1% aqueous TFA) was pipetted several times with the pipette chip to wash away salts remaining in the pipette chip. Finally, proteins were eluted from the pipette chip with 1 μl of a matrix solution (a solution containing 2 mg/ml CHCA, 0.1% TFA, and 70% acetonitrile).

### (3) Mass Spectrometry

1 μl of the eluate containing the proteins was added to a target plate of a MALDI-TOF/TOF mass spectrometer (Applied Biosystems 4700 Proteomics Analyzer, manufactured by Applied Biosystems Inc.) and was left still at normal temperature to crystallize. The MS spectrum and the MS/MS spectrum were measured.

### Example 3

### 3. Data Analysis

The quantitative values for the protein spots determined by the two-dimensional electrophoresis were managed with a laboratory information management system for proteomics (BIOPRISM, manufactured by NEC Co.) and, together with traits data (seven items including individual data and lineage data and 22 items including the quality of meat data), were made into a database. The correlation between data was analyzed.

Furthermore, the MS spectrum and MS/MS spectrum data measured by mass spectrometry were input to MASCOT (Matrix Science Ltd.). Attempts were made to identify the proteins by a peptide mass fingerprint (PMF) analysis or a MS/MS ion search analysis for a public protein sequence database, such as Swiss Prot (http://au.expasy.org/sprot/) or NCBInr (http://au.expasy.org/sprot/). The obtained results were input to the database.

The results of the data analysis of the database showed that protein spots at a pH in the range of 4.0 to 5.0 and a molecular weight in the range of approximately 30 to 40 kDa, specifically, a plurality of protein spots including two encircled protein spots A and B in FIG. 2 were correlated with one of useful traits, carcass weight.

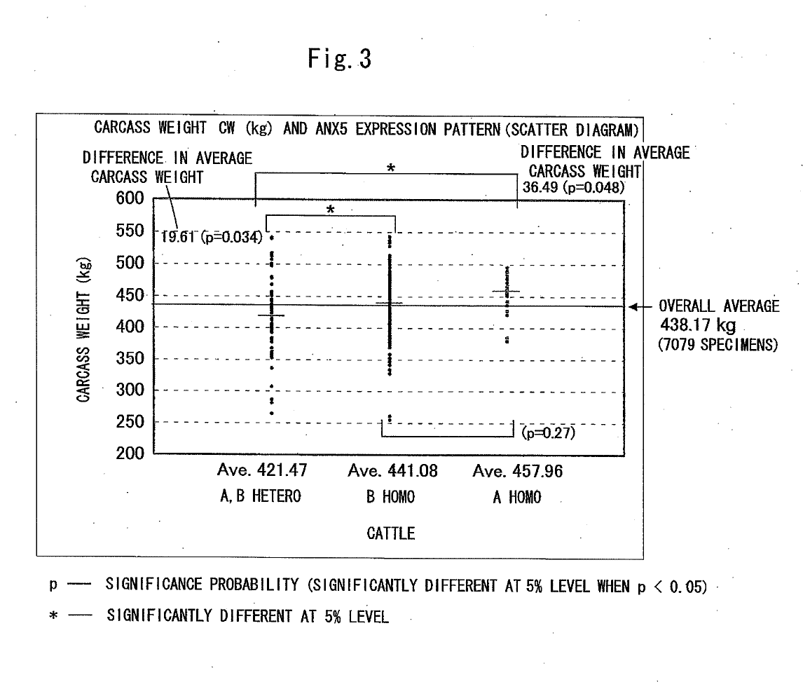

Furthermore, the protein spots at a pH in the range of 4.0 to 5.0 and a molecular weight in the range of approximately 30 to 40 kDa were found to be a wild-type annexin A5 protein (CaBP33) and an isoform thereof (CaBP37). Of the encircled protein spots in FIG. 2, the protein spot A was found to be the isoform (CaBP37) of the wild-type annexin A5 protein (CaBP33), and the protein spot B was found to be the wild-type protein (CaBP33). On the basis of these results, Table 1 and FIG. 3 show the correlation between annexin A5 and carcass weight.

TABLE 1

| | A, B | B | A | |
|---|---|---|---|---|
| Type of annexin A5 | hetero | homo | homo | Total |
| Mix of castrated males and females | 91 | 81 | 14 | 186 |
| (heads) | | | | |
| Castrated males alone (heads) | 70 | 69 | 11 | 150 |
| Average carcass weight of castrated | 421.47 | 441.08 | 457.96 | 438.17 |
| males (kg) | | | | (overall |
| | | | | average) |

In Table 1 and FIG. 3, the comparison of individuals having the wild-type annexin A5 (CaBP33: B) or the isoform thereof A (CaBP37: A) (A homo and B homo in Table 1) with individuals having the wild-type and the isoform thereof (A,B hetero in Table 1) showed a statistically significant difference of approximately 20 to 36 kg (t test, p<0.05) in the average carcass weight of castrated male cattle.

This indicates that the type of annexin A5 included in adipose tissue can be used for investigation to examine the carcass weight of cattle. In other words, this indicates that annexin A5 can be used as a biomarker to examine the carcass weight of cattle.

1. A method for screening cattle, comprising the steps of:
(1) harvesting body tissue from the cattle;
(2) extracting the full complement of proteins from the body tissue harvested;
(3) detecting a wild-type annexin A5 protein, an isoform of the wild-type annexin A5 protein, or a modified protein of the wild-type annexin A5 protein or the isoform thereof in the full complement of proteins extracted; and
(4) determining whether or not the cattle from which the body tissue has been harvested has a high average carcass weight on the basis of whether any one of the wild-type annexin A5 protein and modified protein thereof, an isoform of the wild-type annexin A5 protein and modified protein thereof, or both of them, are detected in the detecting step.

2. The method for screening cattle according to claim 1, wherein the wild-type annexin A5 protein, the isoform of the wild-type annexin A5 protein, and the modified protein of the wild-type annexin A5 protein or the isoform thereof are detected by electrophoresis in the detecting step.

3. The method for screening cattle according to claim 1, wherein the wild-type annexin A5 protein, the isoform of the wild-type annexin A5 protein, and the modified protein of the wild-type annexin A5 protein or the isoform thereof are detected by an antigen-antibody reaction in the detecting step.

4-6. (canceled)

7. A cattle screening kit comprising:
an antibody that specifically bonds to a wild-type annexin A5 protein or a modified protein thereof; and
an antibody that specifically bonds to an isoform of the wild-type annexin A5 protein or a modified protein thereof.
